(19) United States
(12) Patent Application Publication
(10) Pub. No.: US 2014/0336511 A1
(43) Pub. Date: Nov. 13, 2014

(54) ULTRASOUND DIAGNOSIS APPARATUS AND MEDICAL PICTURE PROJECTION APPARATUS

(71) Applicants: KABUSHIKI KAISHA TOSHIBA, Minato-ku (JP); TOSHIBA MEDICAL SYSTEMS CORPORATION, Otawara-shi (JP)

(72) Inventors: Shinichi HOSHINO, Otawara-shi (JP); Junho CHA, Busan (KR); Tomokazu FUJII, Nasushiobara-shi (JP); Yoshihiro OOMORI, Otawara-shi (JP)

(73) Assignees: KABUSHIKI KAISHA TOSHIBA, Minato-ku (JP); TOSHIBA MEDICAL SYSTEMS CORPORATION, Otawara-shi (JP)

(21) Appl. No.: 14/340,119

(22) Filed: Jul. 24, 2014

Related U.S. Application Data
(63) Continuation of application No. PCT/JP2013/073973, filed on Sep. 5, 2013.

(30) Foreign Application Priority Data
Sep. 6, 2012 (JP) 2012-195647
Sep. 5, 2013 (JP) 2013-183692

(51) Int. Cl. A61B 8/08; G09G 5/00

(52) U.S. Cl. CPC A61B 8/5207; G09G 5/006; G09G 2380/08
USPC 600/443; 345/520

(57) ABSTRACT

Ultrasound diagnosis apparatus and a medical picture projection apparatus are provided, simply operating the apparatus in a less loaded posture without losing the variety of operation functions, comprising: an ultrasound probe; signal processor; a display controller; a projector; storage; a determining unit; and a controller. The ultrasound probe scans a subject using ultrasound waves. Signal processor applies signal processing on a first output signal from the ultrasound probe. The display controller causes displaying images based on second output signal from signal processor. The projector projects pictures. Storage preliminarily stores first related information associating an area corresponding to a picture with the operation content among the ultrasound probe, signal processor, the display controller, and the projector. The determining unit determines whether or not at least one part of an object is located within the area indicated in first related information. The controller is based on determination results from determining unit and first related information.

### CROSS-REFERENCE TO RELATED APPLICATION

This application is based upon and claims the benefit of priority from Japanese Patent Applications No. 2012-195647 filed on Sep. 6, 2012 and No. 2013-183692, filed on Sep. 5, 2013; the entire contents of which are incorporated herein by reference.

### FIELD

The embodiments relate to an ultrasound diagnosis apparatus and a medical picture projection apparatus.

### BACKGROUND

Ultrasound diagnosis apparatuses are one type of medical diagnosis apparatuses, and acquire biological information of a subject by transmitting ultrasound waves to the subject using an ultrasound probe and receiving the reflected waves. Those ultrasound diagnosis apparatuses are highly safe, making repetitive use possible for examinations, with a smaller system size in comparison to the medical diagnosis apparatuses, such as X-ray diagnosis apparatuses, X-ray CT (Computed Tomography) apparatuses, or MRI (Magnetic Resonance Imaging) apparatuses, and providing convenience, for example, such that examinations may be performed bed side. Further, an ultrasound diagnosis does not involve radiation exposure by X-rays, enabling usage in obstetrics departments or in home care.

When operating an ultrasound diagnosis apparatus, an operator holds an ultrasound probe with one hand, and positions the ultrasound probe on a predetermined region of a subject. The operator also operates an operation part (a keyboard, a mouse, a touch panel, and the like) of the ultrasound diagnosis apparatus with the other hand to operate the ultrasound diagnosis apparatus. That is, the operator is put in a posture with one hand positioned on the subject side and the other hand on the operation part side.

Further, the operator sometimes holds the ultrasound probe with one hand, and performs a predetermined manipulative procedure with the other hand, depending on test items. For example, in the case of any test items involving milking, the operator performs an ultrasound diagnosis by holding the ultrasound probe with one hand while pressing (milking) the subject's calf with the other hand. Subsequently, the operator operates the ultrasound diagnosis apparatus by operating a foot-operated switch (foot switch) by foot. That is, the operator is put in a posture with one hand at the location of the ultrasound probe and the other hand on the subject's calf while positioning at least one foot on the foot switch side.

Furthermore, X-ray imaging apparatuses, which are one type of the medical diagnosis apparatuses, irradiate X-rays onto a subject and detect the transmitted X-rays for imaging a structure the inside of the subject. For example, an X-ray imaging apparatus performs X-ray imaging in parallel with manipulative procedures, such as catheter insertion, and the like, by an operator (doctor). Subsequently, the operator (doctor) visually confirms images taken by the X-ray imaging apparatus, and performs the manipulative procedures while grasping the internal structure of the subject.

### BRIEF DESCRIPTION OF THE DRAWINGS

FIG. 1 is a block diagram depicting the configuration of an ultrasound diagnosis apparatus in an embodiment.

FIG. 2 is a schematic drawing depicting the outline of the ultrasound diagnosis apparatus in the embodiment.

FIG. 3 is a schematic drawing depicting the outline of the ultrasound diagnosis apparatus in the embodiment.

FIG. 4 is a schematic drawing depicting the outline of the ultrasound diagnosis apparatus in the embodiment.

FIG. 5 is a schematic drawing depicting the outline of the ultrasound diagnosis apparatus in the embodiment.

FIG. 6 is a schematic drawing depicting the outline of the ultrasound diagnosis apparatus in the embodiment.

FIG. 7 is a flowchart depicting the operation of the ultrasound diagnosis apparatus in the embodiment.

FIG. 8 is a block diagram depicting the configuration of an ultrasound diagnosis apparatus of an embodiment.

FIG. 9 is a block diagram depicting the configuration of a medical picture projection apparatus of an embodiment.

### DETAILED DESCRIPTION

An ultrasound diagnosis apparatus is provided capable of simply operating the apparatus in a less loaded posture without losing the variety of operation functions.

Moreover, a medical picture projection apparatus is provided for simply operating a medical diagnosis apparatus in a less loaded posture without losing the variety of operation functions.

### Means for Solving the Problems

An ultrasound diagnosis apparatus of this embodiment comprises: an ultrasound probe; a signal processor; a display controller; a projector; a storage; a determining unit; and a controller.

The ultrasound probe is configured to scan a subject using ultrasound waves. The signal processor is configured to apply signal processing on a first output signal from the ultrasound probe. The display controller is configured to cause a display to display images based on a second output signal from the signal processor. The projector is configured to project pictures. The storage is configured to preliminarily store first related information associating an area corresponding to a picture with the operation content of at least one among the ultrasound probe, the signal processor, the display controller, and the projector.

The determining unit is configured to determine whether or not at least one part of an object is located within the area indicated in the first related information. The controller is configured to conduct controls based on the determination results from the determining unit and the first related information.

A medical picture projection apparatus of this embodiment comprises: an input/output part; a projector; a storage; and a determining unit. The input/output part is configured to be mutually input/output with respect to a medical diagnosis apparatus. The projector is configured to project pictures. The storage is configured to store fourth related information in which the operation content of the medical diagnosis apparatus are associated with an area corresponding to a picture.

The determining unit is configured to determine whether or not at least one part of an object is located within the area indicated in the fourth related information, and output the determination results resulting from the determination to the medical diagnosis apparatus.

### FIRST EMBODIMENT

### Configuration

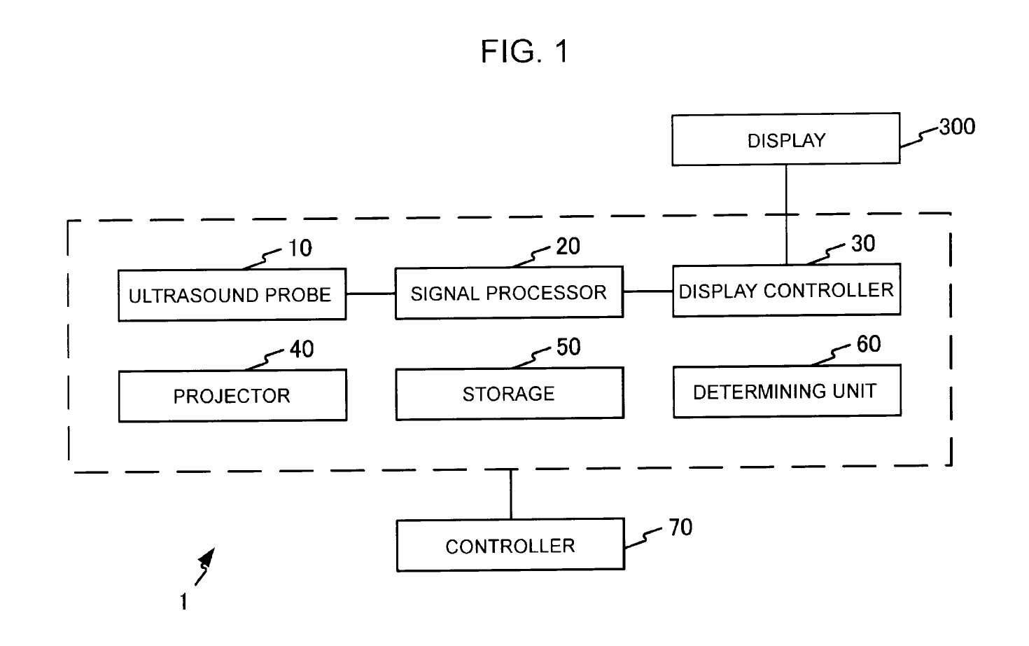

Hereinafter, an ultrasound diagnosis apparatus of this embodiment is described with reference to FIGS. 1 to 7. An ultrasound diagnosis apparatus 1 includes an ultrasound probe 10, a signal processor 20, a display controller 30, a display 300, a projector 40, a storage 50, a determining unit 60, and a controller 70. It should be noted that the display 300 is provided either inside or outside of the ultrasound diagnosis apparatus 1.

(Ultrasound probe 10)

The ultrasound probe 10 scans a subject PA with ultrasound waves. The ultrasound probe 10 uses either a one-dimensional array probe in which a plurality of ultrasound transducers are arranged in one row in the scanning direction or a two-dimensional array probe in which a plurality of ultrasound transducers are arranged two dimensionally. It is also possible to use a mechanical one-dimensional array probe that swings, in the swinging direction perpendicular to the scanning direction, a plurality of ultrasound transducers arranged in one row in the scanning direction.

(Signal processor 20)

The signal processor 20 performs signal processing on a first output signal from the ultrasound probe 10. For example, the ultrasound probe 10 outputs a received echo signal to the signal processor 20 as the first output signal. The signal processor 20 receives the first output signal, performs delay processing thereon, and converts the first output signal to phased data (that is, subjected to reception beamforming).

The signal processor 20 includes, for example, a preamplifier circuit that is not illustrated, an A/D converter, a reception delay circuit, and an adder. The preamplifier circuit amplifies the first output signal output from each ultrasound transducer of the ultrasound probe 10 for each reception channel. The A/D converter converts the amplified echo signal to a digital signal. The reception delay circuit provides a delay time required for determining the reception directionality to the signal converted to the digital signal. The adder adds the signal to which the delay time has been provided. As a result of the addition, a reflective component from a direction corresponding to the reception directionality is enhanced.

The signal processor 20 also includes, for example, a B-mode processor. The B-mode processor visualizes amplitude information of the signal added by the adder. Specifically, the B-mode processor performs band pass filter processing on the signal, subsequently detects the envelope of the signal, and performs compressing processing on the detected data by logarithmic conversion.

The signal processor 20 may also include a CFM (Color Flow Mapping) processor. The CFM processor visualizes blood-flow information. The blood-flow information includes information regarding speed, distribution, power, and the like, and is acquired as binary information.

Moreover, the signal processor 20 may include a Doppler processor. The Doppler processor generates a Doppler frequency distribution that represents blood-flow velocity by removing the Doppler shift frequency component by performing phase detection on signals to perform FFT (Fast Fourier Transform) processing.

The signal processor 20 generates ultrasound image data based on the signals that have been subjected to signal processing (ultrasound raster data). The signal processor 20 includes, for example, a DSC (Digital Scan Converter). The signal processor 20 converts the signals represented by a scanning line signal sequence following the signal processing to image data represented in a perpendicular coordinate system (scan conversion processing). For example, the signal processor 20 generates B-mode image data representing the morphology of an organ of the subject PA by performing scan conversion processing on the signals that has been subjected to the signal processing by the B-mode processor. The signal processor 20 then outputs ultrasound image data to the display controller 30 as a second output signal.

(Display controller 30)

The display controller 30 causes the display 300 to display images based on the second output signal from the signal processor 20. The display controller 30 may also cause the display 300 to display a scanning condition of ultrasound waves by the ultrasound probe 10, a signal processing condition by the signal processor 20, a display condition of images by the display controller 30, medical chart information of the subject PA, and the like. The display 300 is not necessarily included in the ultrasound diagnosis apparatus 1 but may also be configured to display images so as to be controlled by the display controller 300 via a typical interface.

### (Projector 40)

The projector 40 projects pictures. The projector 40 includes, for example, a light source, an optical modulator, a focus lens, and the like. The light source outputs light. The optical modulator modulates the light from the light source based on input picture signals to generate a predetermined picture. The focus lens adjusts the focal position of the light from the optical modulator. As described, the light from the light source is regarded as a picture through the optical modulator, and further projected onto a real space via the focus lens. Regarding the light source, a typical light source, such as an LED (Light Emitting Diode) lamp, a halogen lamp, or the like, may be adopted. Regarding the optical modulator, a typical DMD (Digital Micromirror Device), or the like, may be adopted. The focus lens may be configured such that the position of the focus lens can be moved by a typical drive mechanism, such as a stepping motor, a piezoelectric element, or the like. It should be noted that, for example, the projector 40 is secured onto a couch BE, a ceiling, a floor, or an ultrasound diagnosis apparatus main body by a predetermined arm configured so as to secure the relative positions with respect to the couch or the subject. Subsequently, the projector 40 is secured onto one end of the arm while the other end of the arm is secured onto the couch BE, the ceiling, the floor, or the ultrasound diagnosis apparatus main body. Further, the shape of the arm may be appropriately designed.

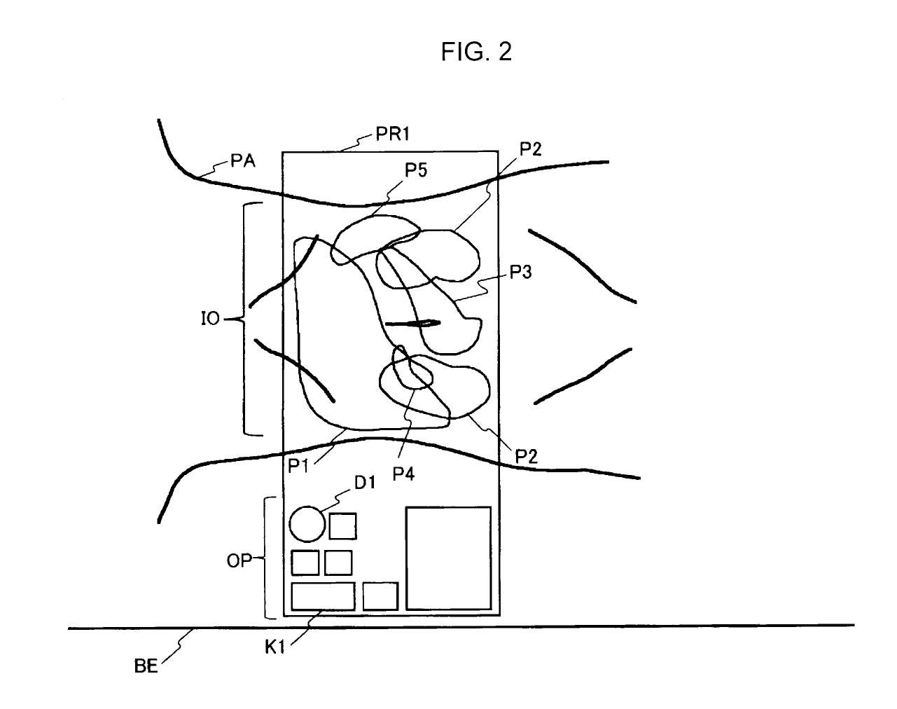

FIG. 2 is a schematic drawing depicting an example of a picture PRI projected by the projector 40. The subject PA is lying on the back on the couch BE. The projector 40 projects the picture PR1 toward the subject PA and the couch BE. The picture PR1 includes an operation part picture OP as well as an organ image IO, the operation part picture OP including a key picture K1 of a schematized key of a keyboard and a dial picture D1 of a schematized dial, the organ image IO including an schematized organ of the subject PA and the schematized location thereof. In FIG. 2, there are pictures schematizing a liver in a picture P1, a kidney in a picture P2, a pancreas in a picture P3, a gallbladder in a picture P4, and a spleen in a picture P5.

It should be noted that, an example of the pictures projected toward the couch BE is shown as one example herein; however, the projector 40 may also project pictures to other locations. For example, the projector 40 may also project pictures to the floor. Further, by preliminarily providing a sheet made from an elastic member, such as rubber, or the like, at a projecting location of the pictures on the couch BE, a floor, or the like, it is also possible to project pictures onto the location where the sheet has been provided.

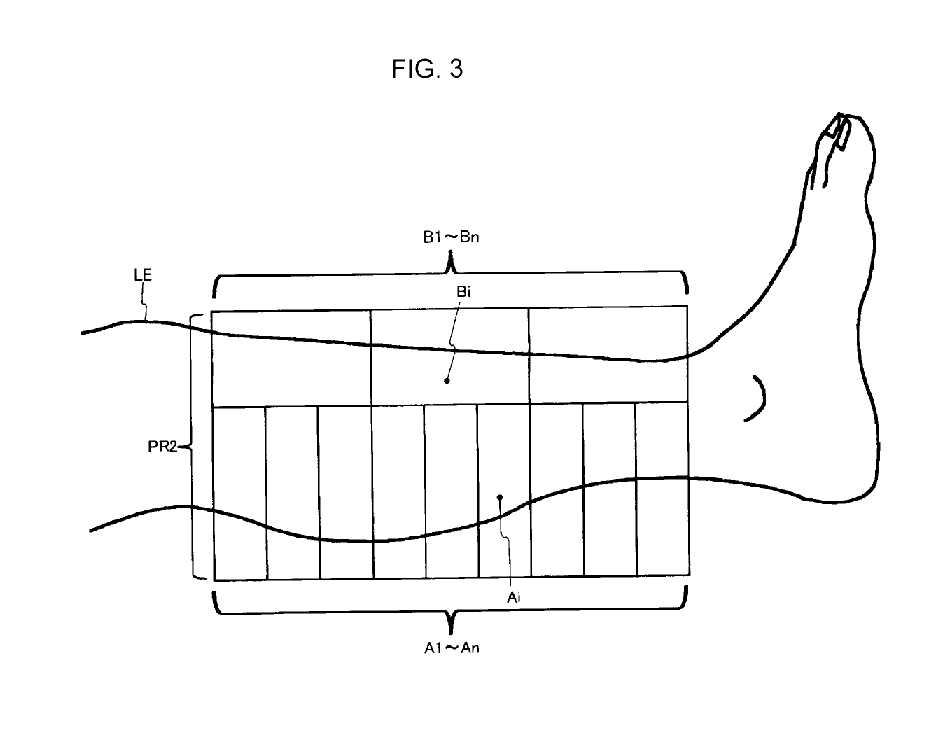

Furthermore, FIG. 3 is a schematic drawing showing another example of pictures projected by the projector 40. The projector 40 projects a picture PR2 toward a lower leg LE of the subject. The picture PR2 is divided into an area Ai (i=1 to n) and an area Bi (i=1 to n).

### (Storage 50)

The storage 50 preliminarily stores first related information in which the operation content of at least one among the ultrasound probe 10, the signal processor 20, the display controller 30, and the projector 40 has been associated with an area corresponding to a picture. The operation content includes at least one among the scanning condition of ultrasound waves by the ultrasound probe 10, the signal processing condition of the first output signal by the signal processor 20, the display condition of images by the display controller 30, and a projection condition of pictures by the projector 40. The scanning condition may include, for example, conditions, such as the focal depth, parallel simultaneous reception number, and the like. The signal processing condition may include a signal processing condition corresponding to the operation mode, such as B-mode, Dopper mode, or the like. The display condition may include contrast or brightness of images, ON/OFF of the freeze display function of images, types of display items, and the like. The projection condition may include types, colors, brightness, and the like, of pictures. Further, the storage 50 may also store, as the first related information, information which associates the operational content with an area corresponding to a picture together with a procedure (protocol) of control by the controller 70, which will be described later.

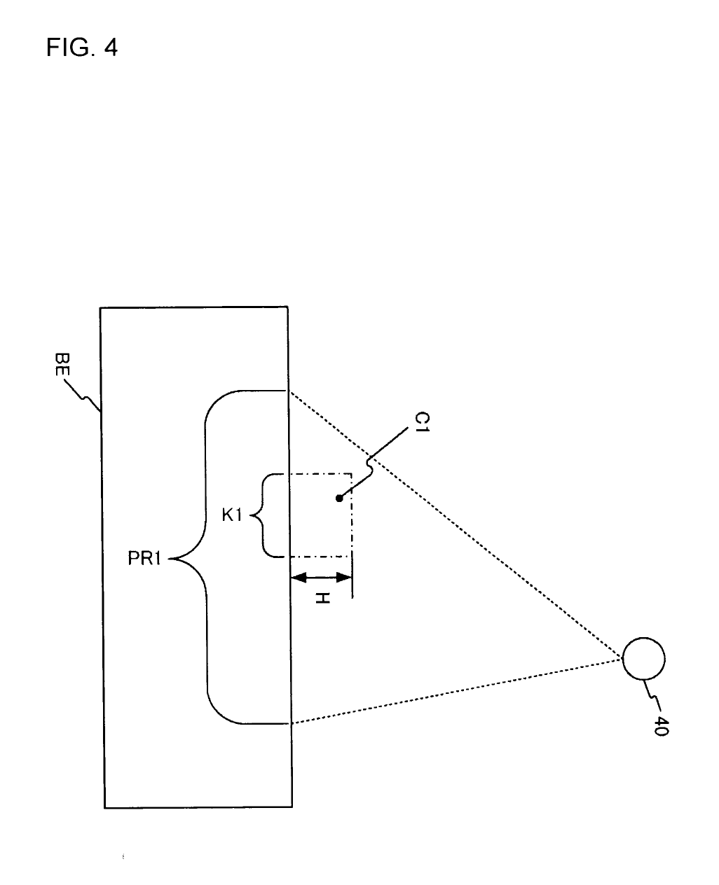

FIG. 4 is a schematic drawing conceptually representing areas corresponding to pictures. FIG. 4 represents the state of the couch BE viewed from the side. Further, for explanation purposes, the subject PA and the pictures P1 to P5 are omitted in FIG. 4. The area corresponding to the picture is, for example, the outer periphery of the key picture P1 within the projected picture PR1 and an area C1 having a predetermined length H from the picture toward the projector 40. This predetermined length H is, for example, preset. Furthermore, this predetermined length Fl may also be determined by the acceptance, in the storage 50, of a designation from an operator DC. As described, the area C1 corresponding to the key picture K1 is determined. Likewise, also with regard to the pictures P1 to P5, the dial picture D1, and the area Ai as well as the area Bi, an area corresponding to each picture is determined.

Moreover, the storage 50 preliminarily stores second related information in which test regions and/or test items are associated with types of pictures. The test regions may include a predetermined organ (e.g. liver, pancreas, or the like) as well as predetermined blood vessels (e.g. carotid arteries, or the like). Further, the test items may include examinations for measuring the internal morphologies of the subject PA and measuring blood-flow movements. The types of pictures may include the arrangement or shape of the organ picture 10 included in the picture PR1 as well as the arrangement or shape of the operation part picture OP. Moreover, this also applies to the area Ai and the area Bi in the picture PR2. It should be noted that in the second related information, schemas (schematic drawings) as a picture may be associated with the test regions and/or the test items. Subsequently, in the second related information, regarding the shape of the organ image IO, schemas (schematic drawings) of each organ are associated with test regions and the test items.

The storage 50 also preliminarily stores third related information in which body-shape information is associated with the sizes of pictures. The body-shape information may include information regarding the height, abdominal girth, chest girth, and the like, of the subject PA. The storage 50 preliminarily stores picture sizes suitable for the body-shape information.

(Determining unit 60)

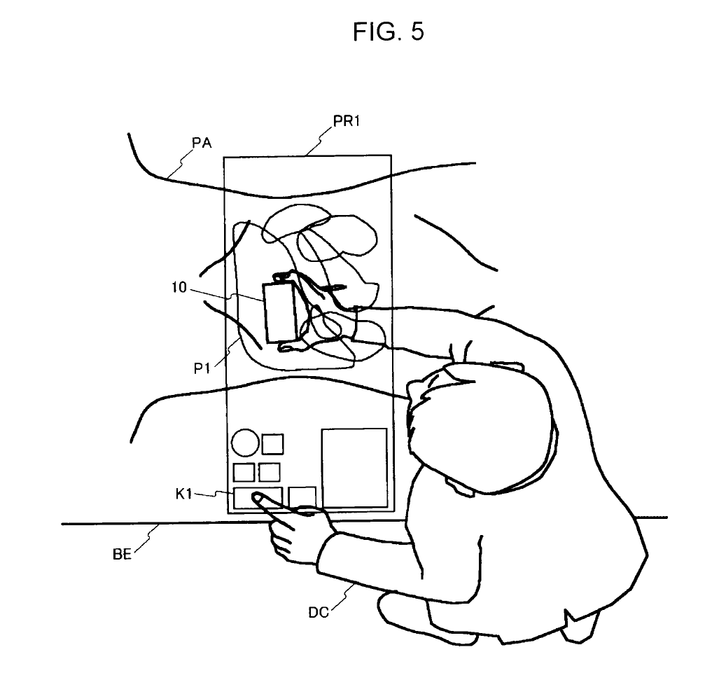

The determining unit 60 determines whether or not at least a part of an object is located within the area indicated in the first related information. The determining unit 60 outputs the determination results according to the determination to the controller 70. Subsequently, in the case that it is determined that at least a part of the object is located within the area indicated in the first related information, the determining unit 60 outputs a signal representing the area to the controller 70. FIG. 5 is a schematic drawing depicting a state in which, in the picture PR1, at least a part of the object is located within the area indicated in the first related information. The object can be, for example, the ultrasound probe 10 or a finger of the operator DC. This determination may also be made by the determining unit 60 by adopting existing technologies used for so-called virtual switches. For example, the determining unit 60 may also make this determination by matching with images taken by an infrared camera or a digital camera. For example, in the case that at least a part of a finger of the operator DC is located in the area of the key picture K1, the determining unit 60 determines that “the object is located in the area of the key picture K1.” It is also possible to configure the determining unit 60 so as to make this determination at a predetermined time interval (for example, at a frame rate of an infrared camera) so that the movement of the object within the predetermined area can be detected. For example, when the finger of the operator DC moves following the circumferential part of the dial picture D1, if the determining unit 60 determines as described above at a predetermined time interval, the determining unit 60 can detect the movement of the finger of the operator DC. This determination is also made in the same way regarding the picture PR2 depicted in FIG. 3.

### (Controller 70)

The controller 70 conducts controls based on the determination results by the determining unit 60 and the first related information. For example, when at least a part of an object is located in the area indicated in the first related information, the controller 70 controls each part of the ultrasound diagnosis apparatus based on the operation content associated with the area. For example, the controller 70 reads, from the storage 50, the operation content with which the area represented in the signal received from the determining unit 60 is associated in the first related information. The controller 70 conducts controls based on the read operation content. For example, when the scanning condition of ultrasound waves by the ultrasound probe 10 is included in the read operation content, the controller 70 controls the ultrasound probe 10 so that the ultrasound probe 10 operates based on the read scanning condition. Further, when the signal processing condition by the signal processor 20 is included in the read operation content, the controller 70 controls the signal processor 20 so that the signal processor 20 operates based on the read signal processing condition. Furthermore, when the display condition by the display controller 30 is included in the read operation content, the controller 70 controls the display controller 30 so that the display controller 30 operates based on the read display condition. Moreover, when the projection condition by the projector 40 is included in the read operation content, the controller 70 controls the projector 40 so that the projector 40 operates based on the read projection condition.

For example, in the first related information, when the area of the key picture K1 has been associated with the operation content of image freezing ON, if the determining unit 60 determines that an object, such as a finger of the operator DC, or the like, is located in the area of the key picture K1, the controller controls the display controller 30 to freeze the images (still images are displayed). Further, in the first related information, when the area of the picture P1 (schematic drawing of a liver) has been associated with the operation content for examining a liver, if the determining unit 60 determines that at least a part of an object such as the ultrasound probe 10, or the like, is located in the area of the picture P1, the controller 70 controls at least one among the ultrasound probe 10, the signal processor 20, and the display controller 30 to set at least one condition among the scanning condition, the signal processing condition, and the display condition associated with the area of the picture P1, as the operation content for examining the liver. Furthermore, in the storage 50, when the information associated with the picture P1 together with a procedure (protocol) for examining the liver as the operation content for examining the liver has been stored as the first related information, the controller 70 may control the operation content of each part based on this procedure. The controller 70 may also control the display controller 30 to cause the display 300 to display a predetermined confirmation screen, and control the operation content of each part based on this procedure after receiving an instruction to start the protocol from the operator DC.

It should be noted that the controller 70 may also be configured so as to appropriately conduct this control in accordance with the progress of examination procedures. In an ultrasound diagnosis, the operator DC moves the tip end part of the ultrasound probe 10 so as to slide over the body surface of the subject PA. At this time, it is not required for the controller 70 to change the operation content every time the movement of the ultrasound probe 10 is detected by the determining unit 60. For example, the controller 70 may be configured so as to appropriately switch ON/OFF control functions of the operation content by the controller 70 after receiving an input operation by the operator DC.

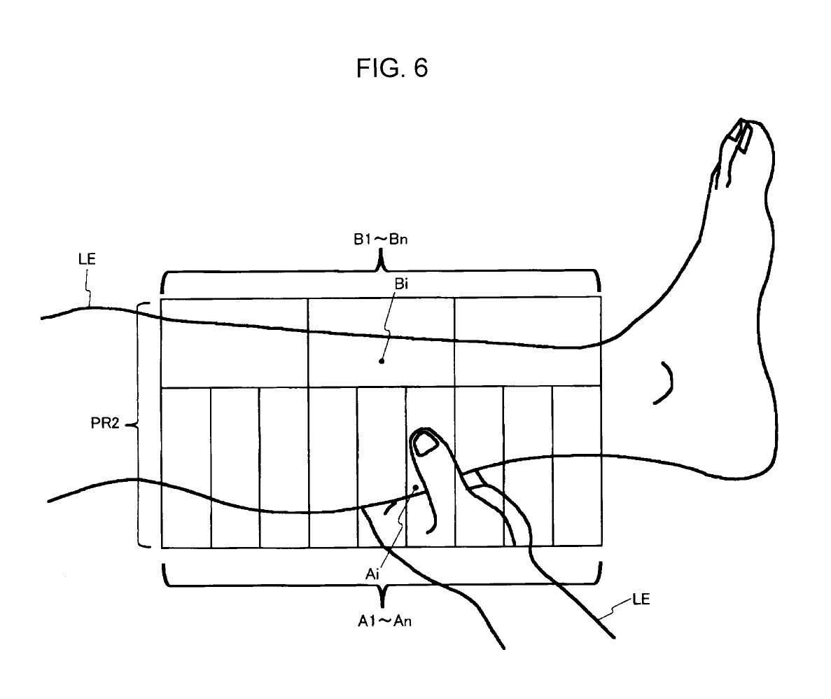

FIG. 6 is a schematic drawing depicting a state in which, in the picture PR2, at least a part of an object is located within the area indicated in the first related information. For example, it is provided that the area An is associated with a predetermined scanning condition and the area Bn is associated with the operation content of image freezing ON. If the determining unit 60 determines that a fingertip of the operator DC, or the like is located in the area An (if the operator DC performs milking for a part in the area An of the lower leg part LE), the controller 70 controls the ultrasound probe 10 and makes a change to the predetermined scanning condition. Further, when it is determined that the fingertip of the operator DC, or the like is located in the area Bn (if the operator DC causes the fingertip to be located in the area Bn), the controller 70 controls the display controller 30 to freeze the images (still images are displayed).

Furthermore, the controller 70 may also be configured so as to move the operation part picture OP to a preferable location by controlling the projection condition of the projector 40. For example, the operator DC is capable of arranging the key picture K1 and the dial picture D1 of the operation part picture OP to preferable locations by tracing the same with his finger. Moreover, the operator DC is also capable of similarly moving the position of the image to arrange the organ image 10 in a location suitable for examination.

Furthermore, upon receipt of a designation of test regions and/or an test items, the controller 70 causes the projector 40 to project pictures associated with the test regions and/or the test items. For example, when a kidney is designated as the test region during examining a kidney, the controller 70 causes the projector 40 to project the picture P2 associated with the kidney (a schematic drawing of the kidney). At this time, the controller 70 may also cause the projector 40 to project an operation part picture OP that is suitable for the kidney examination. It should be noted that this designation may also be performed with regard to all of the test regions and/or the test items involving the procedure of the examination at the time when the examination starts. Further, this designation may also be performed consecutively during the examination. Moreover, this designation may also be performed by the operator DC by a publically known operating means (not illustrated), such as a keyboard, or the like. Furthermore, in the second related information, when a schema as a picture is associated with a test region and/or a test item, the controller 70 causes the projector 40 to project the schema associated with the designated test region and/or test item. Thereby, the projector 40 projects, as the organ image IO, a schema of an organ that is the designated test region or a schema of an organ that is an object of the designated test item.

The controller 70 also causes the projector 40 to project a picture based on the body-shape information of the subject PA and the third related information. The controller 70 receives the designation of the body-shape information of the subject PA, and causes the projector 40 to project a picture of the size that associated in the third related information. This designation may be conducted by the operator DC by a publicly known operating means (not illustrated), such as a keyboard, or the like.

### Operation

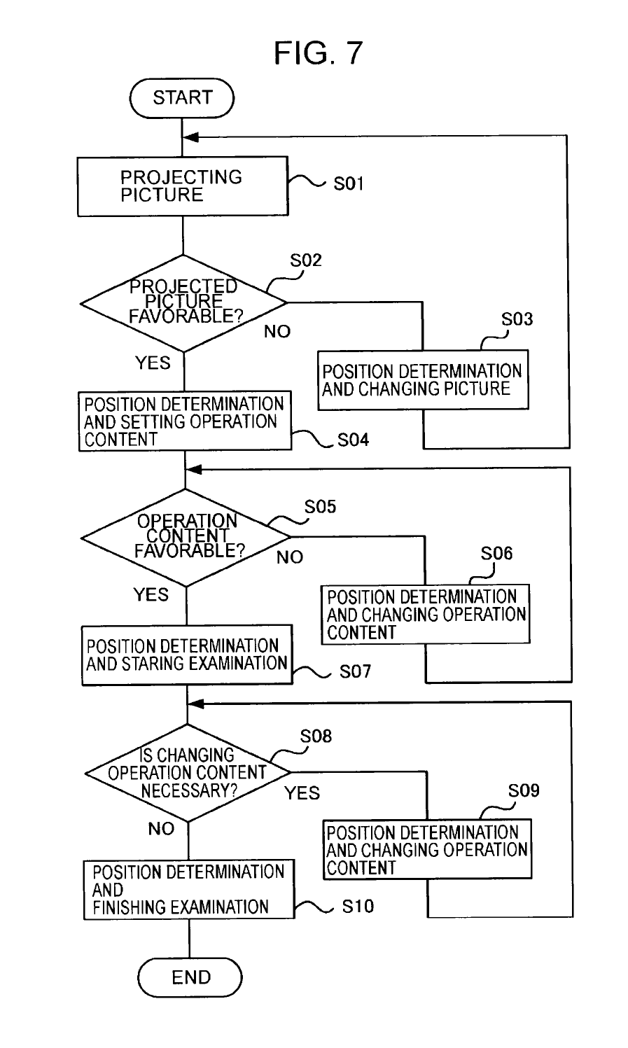

An operation example of the ultrasound diagnosis apparatus in this embodiment is described with reference to the flowchart in FIG. 7.

### (S01)

Upon receipt of a designation of area test region and/or a test item, the controller 70 controls the projector 40 to cause the projector 40 to project the picture PR1 that has been associated with the test region and/or the test item. Further, the controller 70 controls the projector 40 based on the body-shape information of the subject PA as well as the third related information, to cause the projector 40 to project the picture PR1.

### (S02, S03)

When the projected picture PR1 is not favorably suited for the examination (S02:NO), for example, the operator DC traces the key picture K1 in the operation part picture OP with a finger so as to move and arrange the key picture K1 in a desired location. At this time, the determining unit 60 determines that “an object is located in the area of the key picture K1.” The controller 70 controls the projector 40 based on the determination results by the determining unit 60 and the first related information so as to change the projection condition of the key picture K1 as the operation content of the projector 40. Thereby, the key picture K1 is projected onto the location desired by the operator DC (S03). This applies to other pictures, such as the dial picture D1, and the like.

### (S02, S04)

When the projected picture PR1 is favorably suitable for the examination (S02:YES), and when the operator DC positions the ultrasound probe 10 on the test region (the area of the picture depicting the test region), the determining unit 60 determines that “an object is located in the area of the picture depicting the test region.” The controller 70 conducts controls based on the determination results from the determining unit 60 and the first related information. That is, at least one of the operation content among the scanning condition with ultrasound waves by the ultrasound probe 10, the signal processing condition of a first output signal by the signal processor 20, the display condition of images by the display controller 30, and the projection condition of the picture PR1 by the projector 40 is set (S04).

### (S05, S06)

When the set operation content is not favorably suitable for the examination (S05:NO), the operator DC positions a finger in an arbitrary area in the operation part picture OP to change the operation content. At the time, the determining unit 60 determines whether or not at least a part of the object within the area indicated in the first related information is located. The determining unit 60 then outputs the determination results from the determination to the controller 70. The controller 70 controls at least one among the ultrasound probe 10, the signal processor 20, and the display controller 30 to change at least one condition, as the operation content, among the scanning condition, the signal processing condition, and the display condition, based on the determination results from the determining unit 60 and the first related information (S06).

### (S05, S07)

When the set operation content is favorably suited for the examination (S05:YES), the operator DC positions a finger in the area associated with a function to start an examination in the operation part picture OP to begin the examination. At this time, the determining unit 60 determines that “an object is located in the area.” The determining unit 60 then outputs the determination results by this determination to the controller 70. The controller 70 controls the ultrasound probe 10 based on the determination results from the determination part 60 and the first related information to scan the subject PA with ultrasound waves. The controller 70 also controls the signal processor 20 based on the determination results from the determination part 60 and the first related information to implement signal processing on a first output signal from the ultrasound probe 10. Further, the controller 70 controls the display controller 30 based on the determination results from the determination part 60 and the first related information to cause the display part 300 to display images based on a second output signal from the signal processor 20 (S07).

### (S08, S09)

When changes to the operation content are required during the examination (S08:YES), the operator DC positions a finger in an arbitrary area in the operation part picture OP to change the operation content. At this time, the controller 70 changes the operation content based on the determination results from the determining unit 60 and the first related information. The determining unit 60 determines whether or not at least a part of an object is located in the area indicated in the first related information. The determining unit 60 then outputs the determination results by this determination to the controller 70. The controller 70 controls at least one among the ultrasound probe 10, the signal processor 20, and the display controller 30 to change at least one condition, as the operation content, among the scanning condition, the signal processing condition, and the display condition based on the determination results from the determining unit 60 and the first related information (S09).

### (S08, S10)

When changes to the operation content are not required during the examination (S08:NO), the operator DC positions a finger in the area associated with the function to start an examination in the operation part picture OP to end the examination. The controller 70 then finishes the examination based on the determination results from the determining unit 60 and the first related information (S10). Hereinabove, the operations depicted in FIG. 7 are finished.

### Operation and Effect

Operations and effects of the ultrasound diagnosis apparatus in this embodiment are described.

The ultrasound diagnosis apparatus 1 includes the ultrasound probe 10, the signal processor 20, the display controller 30, the projector 40, the storage 50, the determining unit 60, and the controller 70. The ultrasound probe 10 scans the subject PA with ultrasound waves. The signal processor 20 applies signal processing on a first output signal from the ultrasound probe 10. The display controller 30 causes the display 300 to display images based on a second output signal from the signal processor 20. The projector 40 projects pictures. The storage 50 preliminarily stores the first related information in which the operation content of at least one among the ultrasound probe 10, the signal processor 20, the display controller 30, and the projector 40 has been associated with an area in a picture. The determining unit 60 determines whether or not at least a part of an object is located within the area indicated in the first related information. The controller 70 conducts controls based on the determination results from the determining unit 60 and the first related information. By such a configuration, the operator DC is able to operate the ultrasound diagnosis apparatus 1 using an area corresponding to a picture and the location of the object. Thereby, it becomes possible to provide an ultrasound diagnosis apparatus simply operatable by the operator DC in a less loaded posture without losing the variety of operational functions.

Further, the operation content of the ultrasound diagnosis apparatus 1 may include at least one among the scanning condition with ultrasound waves by the ultrasound probe 10, the signal processing condition of a first output signal by the signal processor 20, the display condition of images by the display controller 30, and the projection condition of pictures by the projector 40. As described above, the storage 50 stores the first information in which the operation content has been associated with an area within a picture. The controller 70 then performs control on at least one part of the operation content based on the determination results from the determining unit 60 and the first related information. That is, setting may be possible for the operator DC with regard to at least one part of the operation content via the picture. Thereby, it becomes possible to provide an ultrasound diagnosis apparatus that can be operated more simply in a less loaded posture.

Furthermore, in the ultrasound diagnosis apparatus 1, the storage 50 preliminarily stores the second related information in which test regions and/or test items have been associated with the types of pictures, upon receipt of a designation of the test regions and/or the test items, the controller 70 may cause the projector 40 to project the pictures associated with the test regions and/or the test items. In this way, the projector 40 can project pictures corresponding to the test regions and/or the test items by designating the test regions and/or the test items. Moreover, in the second related information stored in the storage 50, schemas as a picture may be associated with test regions and/or test items and the controller 70 may cause the projector 40 to project the schemas associated with the designated test regions and/or test items. Thereby, it becomes possible to provide an ultrasound diagnosis apparatus that can be operated more simply without losing the variety of operational functions.

Moreover, in the ultrasound diagnosis apparatus 1, the storage 50 may preliminarily store the third related information in which the body-shape information has been associated with the sizes of pictures, and the controller 70 may also cause the projector 40 to project the pictures based on the body-shape information of the subject PA and the third related information. In this way, the projector 40 may project pictures at sizes corresponding to the body shape of the subject PA. Thereby, it becomes possible to provide an ultrasound diagnosis apparatus that can be operated more simply.

### SECOND EMBODIMENT

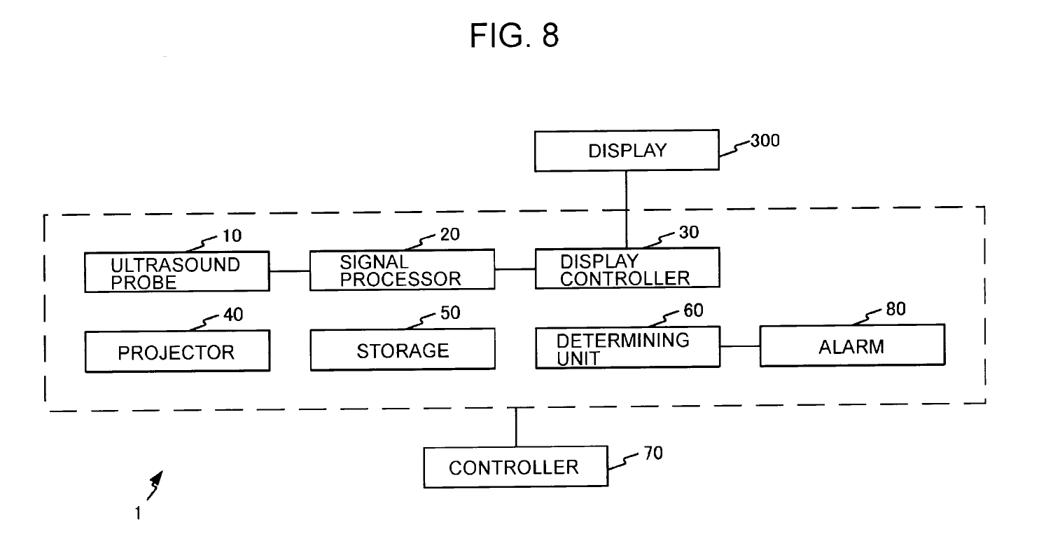

Hereinafter, an ultrasound diagnosis apparatus of this embodiment is described with reference to FIG. 8. The ultrasound diagnosis apparatus in the embodiment includes an alarm 80. Other configurations are the same as in the first embodiment.

### Configuration

The alarm 80 demonstrates determination results based on the determination results from the determining unit 60. For example, in the case that the alarm 80 determines that at least a part of an object is located within an area indicated in the first related information, the alarm 80 demonstrates the determination results from the determining unit 60 by changing the color of the picture of the area. Furthermore, when demonstrated, the determination results from the determining unit 60 may also be demonstrated by the alarm 80 with a beeping sound.

### Operation and Effect

Operations and effects of the ultrasound diagnosis apparatus of this embodiment are described.

The alarm 80 demonstrates determination results based on the determination results from the determining unit 60. Thereby, the operator DC is able to simply know whether or not at least a part of an object is located within an area indicated in the first related information. Therefore, it is possible to provide an ultrasound diagnosis apparatus that can be operated more simply.

### THIRD EMBODIMENT

### Configuration

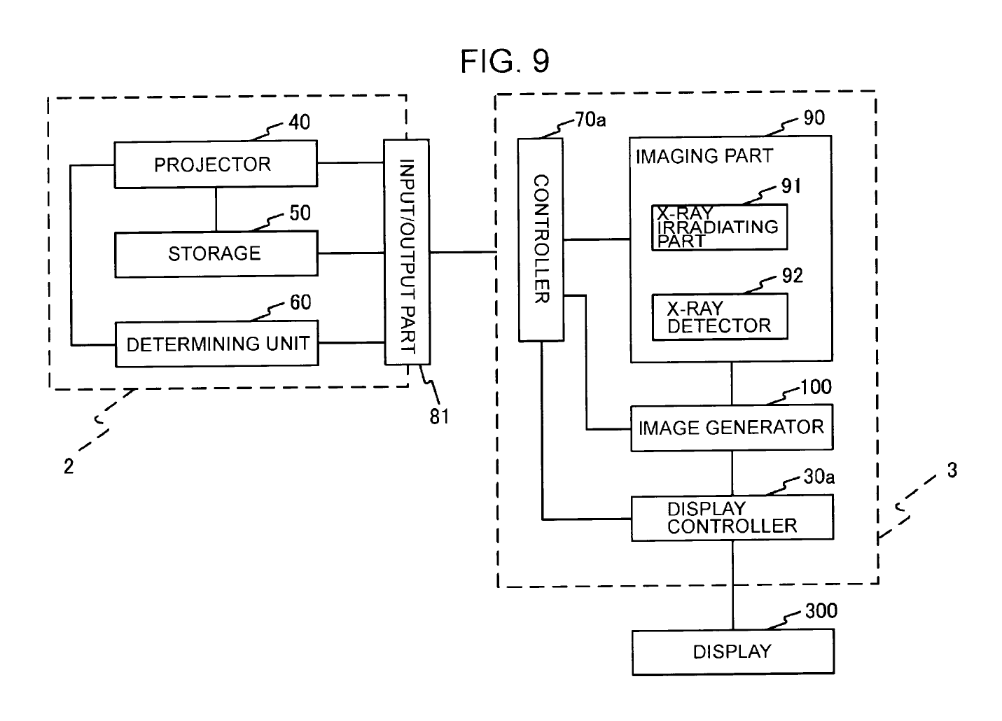

FIG. 9 is a block diagram representing the configuration of a medical picture projection apparatus 2 of this embodiment. The medical picture projection apparatus 2 includes an input/output part 81, the projector 40, the storage 50, and the determining unit 60. A medical diagnosis apparatus 3 includes an imaging part 90, an image generator 100, a display controller 30a, the display 300, and a controller 70a. The medical picture projection apparatus 2 in the embodiment is configured as a separate body from the medical diagnosis apparatus 3. It should be noted that the display 300 is provided either inside or outside of the medical diagnosis apparatus 3. In the embodiment, a case in which the medical diagnosis apparatus 3 is an X-ray imaging apparatus is described. Hereinafter, in some cases, a description is omitted regarding the same items as in the first embodiment.

The imaging part 90 includes an X-ray irradiating part 91 and an X-ray detector 92. The X-ray irradiating part 91 irradiates X-rays onto the subject PA. The X-ray irradiating part 91 includes an X-ray tube. The X-ray irradiating part 91 also includes an inverter and a high pressure transformer which have not been illustrated. The X-ray tube generates X-rays. The X-ray tube includes an electronic gun consisting of a filament and a target. The X-ray irradiating part 91 applies a high voltage between the filament and the target via the inverter and the high voltage transformer to cause electrons jumped out from the filament to collide with the target, in order to generate X-rays from the X-ray tube. Subsequently, the X-ray irradiating part 91 irradiates the generated X-rays onto the subject PA. The operation content as an imaging condition of the imaging part 90 involves a tube current or a tube voltage of the X-ray tube or both.

The X-ray detector 92 detects X-rays transmitted through the subject PA. The X-ray detector 92 is configured including a plurality of X-ray detecting elements. The X-ray detector 92 detects, by the X-ray detecting elements, X-ray intensity distribution data indicating the intensity distribution of X-rays transmitted through the subject PA, and outputs the X-ray intensity distribution data to the image generator 100.

The image generator 100 consecutively generates a plurality of X-ray images at a fixed time interval (frame rate) based on the X-ray intensity distribution data from the X-ray detector 92. Herein, an image generated by the image generator 100 based on the detection results at one time phase is regarded as one X-ray image (frame). The image generator 100 generates image data corresponding to each of a plurality of time phases based on the detection results at each of the plurality of time phases. That is, the image generator 100 consecutively generates X-ray images by mapping the X-ray images to the time phases. It should be noted that in this generating process, the X-ray intensity distribution data is subjected to off-set correction, X-ray intensity correction, defects pixel correction, and the like, by the image generator 100. The operation content includes, as an image generating condition by the image generator 100, frame rate, an off-set correction condition, an X-ray intensity correction condition or a defect pixel correction condition, or a combination of these. The image generator 100 then consecutively outputs the generated image data to the display controller 30a.

The display controller 30a causes the display 300 to display images based on the image data from the image generator 100. The display controller 30a may also cause the display 300 to display imaging conditions by the imaging part 90, the image generating conditions by the image generator 100, and medical chart information of the subject PA, and the like. The operation content includes a display condition of images by the display controller 30a.

The image controller 70a conducts controls based on the determination results from the determining unit 60 and the first related information. For example, when at least a part of an object is located in the area indicated in the first related information, the controller 70a controls each part of the medical diagnosis apparatus 3 based on the operation content associated with the area. For example, the controller 70a reads, from the storage 50, the operation content associated with the area represented in a signal from the determining unit 60 in the first related information. The controller 70a conducts controls based on the read operation content. For example, when the imaging condition by the imaging part 90 is included in the read operation content, the controller 70a controls the imaging part 90 so that the imaging part 90 operates based on the read imaging condition. Further, when the image generating condition by the image generator 100 is included in the read operation content, the controller 70a controls the image generator 100 so that the image generator 100 operates based on the read image generating condition. Furthermore, when the display condition by the display controller 30a is included in the read operation content, the controller 70a controls the display controller 30a so that the display controller 30a operates based on the read display condition. Moreover, upon receipt of a designation of test regions and/or test items, the controller 70a outputs a signal representing the designated regions and/or test items to the projector 40.

The input/output part 81 is capable of mutually inputting//outputting to/from the medical diagnosis apparatus 3. For example, the input/output part 81 is configured as an interface based on predetermined communication standard, such as RS-232C, IEE-488, USB, or the like, whereby, each part of the medical picture projection apparatus 2 and each part of the medical diagnosis apparatus 3 are connected so as to be mutually input/output.

The projector 40 projects pictures. The projector 40 receives a signal representing test regions and/or test items from the controller 70a. The projector 40 reads, from the storage 50, pictures associated with the test regions and/or the test items represented in the signal. The projector 40 projects the read pictures. For example, the projector 40 projects pictures toward the couch BE. The pictures include an operation part picture OP including a key picture K1 of schematized key on a keyboard and a dial picture D1 of a schematized dial.

The storage 50 preliminarily stores fourth related information in which the operation content of the medical diagnosis apparatus 3 has been associated with an area corresponding to a picture. For example, the storage 50 preliminarily receives an input of the fourth related information in accordance with the medical diagnosis apparatus 3. The storage 50 only has to preliminarily store the fourth related information in accordance with the medical diagnosis apparatus 3. Therefore, a subject for inputting the fourth related information into the storage 50 is not limited. In the case that the medical diagnosis apparatus 3 is an X-ray imaging apparatus, the operation content includes at least one among the imaging condition by the imaging part 90, the image generating condition by the image generator 100, the display condition by the display controller 30a, and the projection condition by the projector 40. Furthermore, the storage 50 preliminarily stores second related information in which test regions and/or test items have been associated with the types of pictures. For example, the storage 50 preliminarily receives an input of the second information in accordance with the medical diagnosis apparatus 3. The storage 50 only has to preliminarily store the second related information in accordance with the medical diagnosis apparatus 3. Therefore, a subject for inputting the second related information into the storage 50 is not limited.

The determining unit 60 determines whether or not at least a part of an object is located within the area indicated in the fourth related information. The determining unit 60 outputs the determination results from the determination to the medical diagnosis apparatus 3. When it is determined that at least a part of the object is located within the area indicated in the fourth related information, the determining unit 60 outputs a signal representing the area to the controller 70a.

### Operation and Effect

Operations and effects of the medical picture projection apparatus 2 in this embodiment are described.

The medical picture projection apparatus 2 includes the input/output part 81, the projector 40, the storage 50, and the determining unit 60. The input/output part 81 is connected to the medical diagnosis apparatus 3 so as to be mutually input/output. The projector 40 projects pictures. The storage 50 preliminarily stores the fourth related information in which the operation content of the medical diagnosis apparatus 3 has been associated with the area corresponding to the picture. The determining unit 60 determines whether or not at least a part of an object is located within the area indicated in the fourth related information, and outputs the determination results from the determination to the medical diagnosis apparatus 3. In this way, the operator DC can operate the medical diagnosis apparatus 3 using the area corresponding to the pictures projected by the medical picture projection apparatus 2 and the location of the object. Thereby, it becomes possible to provide the medical picture projection apparatus 2 for simply operating the medical diagnosis apparatus 3 in a less loaded posture without losing the variety of operational functions.

### COMMON EFFECTS AMONG THE EMBODIMENTS

According to the ultrasound diagnosis apparatus or the medical picture projection apparatus of at least one of the embodiments described above, it becomes possible to project pictures for simply operating a medical diagnosis apparatus in a less loaded posture without losing the variety of operational functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasound diagnosis apparatus, comprising:
an ultrasound probe configured to scan a subject using ultrasound waves;
a signal processor configured to apply signal processing on a first output signal from the ultrasound probe;
a display controller configured to cause a display to display images based on a second output signal from the signal processor;
a projector configured to project pictures;
a storage configured to preliminarily store first related information associating an area corresponding to a picture with the operation content of at least one among the ultrasound probe, the signal processor, the display controller, and the projector;
a determining unit configured to determine whether or not at least one part of an object is located within the area indicated in the first related information; and
a controller configured to conduct controls based on the determination results from the determining unit and the first related information.

2. The ultrasound diagnosis apparatus according to claim 1, wherein the operation content includes at least one among a scanning condition of ultrasound waves by the ultrasound probe, a signal processing condition of the first output signal by the signal processor, a display condition of images by the display controller, and a projection condition of pictures by the projector.

3. The ultrasound diagnosis apparatus according to claim 1, wherein
the storage is configured to preliminarily store second related information in which test regions and/or test items are associated with picture types, and
the controller is configured to receive a designation of test regions and/or test items, and cause the projector to project pictures associated with the test regions and/or the test items.

4. The ultrasound diagnosis apparatus according to claim 3, wherein
the storage is configured to preliminarily store third related information associating body-shape information with picture sizes, and
the controller is configured to cause the projector to project pictures based on the body-shape information of the subject and the third related information.

5. The ultrasound diagnosis apparatus according to claim 3, wherein in
in the second related information, schemas in the form of the pictures are associated with the test regions and/or the test items, and
the controller is configured to cause the projector to project the schemas associated with the designated test regions and/or test items.

6. The ultrasound diagnosis apparatus according to claims 1, further comprising, based on the determination results of the determining part, an alarm configured to alarm the determination results.

7. A medical picture projection apparatus, comprising:
an input/output part configured to be mutually input/output with respect to a medical diagnosis apparatus;
a projector configured to project pictures;
a storage configured to store fourth related information in which the operation content of the medical diagnosis apparatus are associated with an area corresponding to a picture, and
a determining part configured to determine whether or not at least one part of an object is located within the area indicated in the fourth related information, and output the determination results resulting from the determination to the medical diagnosis apparatus.

8. The ultrasound diagnosis apparatus according to claim 2, further comprising, based on the determination results of the determining part, an alarm configured to alarm the determination results.

9. The ultrasound diagnosis apparatus according to claim 3, further comprising, based on the determination results of the determining part, an alarm configured to alarm the determination results.

10. The ultrasound diagnosis apparatus according to claim 4, further comprising, based on the determination results of the determining part, an alarm configured to alarm the determination results.

11. The ultrasound diagnosis apparatus according to claim 5, further comprising, based on the determination results of the determining part, an alarm configured to alarm the determination results.
